(19) United States
(12) Patent Application Publication
(10) Pub. No.: US 2026/0284249 A1
(43) Pub. Date: Sep. 24, 2026

(54) Disinfecting Cap

(71) Applicant: Medline Industries, LP, Northfield, IL (US)

(72) Inventors: Yushek Pun, Dayton, NJ (US); Joseph Guerrero, Edison, NJ (US); Alexander Redfield, Carneys Point, NJ (US); Bridget Kelley, Chicago, IL (US); William Kyle Hyland, Oceanport, NJ (US)

(21) Appl. No.: 19/691,328

(22) Filed: May 28, 2026

Related U.S. Application Data
(63) Continuation-in-part of application No. 17/981,328, filed on Nov. 4, 2022.
(60) Provisional application No. 63/276,195, filed on Nov. 5, 2021.
(60) Provisional application No. 63/814,036, filed on May 29, 2025.

(51) Int. Cl. A61L 2/26; A61L 2/18; A61L 103/15

(52) U.S. Cl. CPC A61L 2/26; A61L 2/18; A61L 2103/15; A61L 2202/121; A61L 2202/123

(57) ABSTRACT

A device for cleaning medical implements is disclosed, in particular a device for cleaning vascular or other fluid access sites. The device includes a cap having an opening to receive an access site. The cap may be used in the following manner: A healthcare worker may, with gloved hands, open the cap packaging and place the cap over the port of a medical implement to be cleaned. The healthcare worker may wipe the site by either applying a turning motion or by simply pushing the cap onto the port. A cap in place on a medical implement may be a positive indication that a desired site of the medical implement is clean. The cap may include a disinfecting substance. Embodiments of the cap may comprise a cap body and a skirt.

### CROSS REFERENCE TO EARLIER APPLICATIONS

This application claims priority to and benefit of U.S. Provisional Patent Application No. 63/814,036, filed on May 29, 2025. In addition, this application is a continuation-in-part of U.S. patent application Ser. No. 17/981,328, filed on Nov. 4, 2022, which application claims priority to and benefit of U.S. Provisional Patent Application No. 63/276,195, filed on Nov. 5, 2021. Each of these applications is incorporated herein by reference.

### BACKGROUND

Within the medical field, and in particular the area of infusion of fluids or aspiration of fluids to or from a patient, there is a need to prevent the transmission of pathogens into or onto a patient from a potentially contaminated surface of a medical implement. Such pathogens include microorganisms such as bacteria and viruses. The transmission of pathogens into a patient may result in an infection that could be life threatening. Common sites for such transmissions are found at access “sites” of medical implements such as a luer port, vial, needle free valve, or an injection port of a vessel, tubing, or catheter. Even non-intrusive medical implements such as stethoscopes or otoscopes can transmit pathogens to a patient.

Accordingly, a need exists for an apparatus and technique for cleaning a site on a medical implement prior to contact with a patient.

### BRIEF DESCRIPTION OF THE DRAWINGS

Advantages of the invention will become apparent upon reading the following detailed description and upon reference to the drawings.

FIG. 1 is a perspective view of a disinfecting cap exemplary embodiment showing a closed end of the cap.

FIG. 2 is a perspective view of the disinfecting cap of FIG. 1 showing an open end of the cap.

FIG. 3 shows a cross-sectional view of the disinfecting cap of FIGS. 1-2.

FIG. 4 is a perspective view of a disinfecting cap exemplary embodiment showing a closed end of the cap.

FIG. 5 is a perspective view of the disinfecting cap of FIG. 4 showing an open end of the cap.

FIG. 6 shows a cross-sectional view of the disinfecting cap of FIGS. 4-5.

FIG. 7 is a perspective view of a disinfecting cap exemplary embodiment showing an open end of the cap.

FIG. 8 is a side view of the disinfecting cap of FIG. 7.

FIG. 9 is a cross-sectional view of the disinfecting cap of FIG. 8 along line A-A.

FIG. 10 is an end view of the disinfecting cap of FIG. 7 showing an open end.

FIG. 11 is an end view of the disinfecting cap of FIG. 7 showing a closed end.

FIG. 12 is a perspective view of a disinfecting cap exemplary embodiment showing an open end of the cap.

FIG. 13 is a side view of the disinfecting cap of FIG. 12.

FIG. 14 is a cross-sectional view of the disinfecting cap of FIG. 13 along line A-A.

FIG. 15 is an end view of the disinfecting cap of FIG. 12 showing an open end.

FIG. 16 is an end view of the disinfecting cap of FIG. 12 showing a closed end.

FIG. 17 is a perspective view of a disinfecting cap exemplary embodiment showing a closed end of the cap.

FIG. 18 is a perspective view of the disinfecting cap of FIG. 17 showing an open end of the cap.

FIG. 19 is a side view of the disinfecting cap of FIG. 17.

FIG. 20 is a cross-sectional view of the disinfecting cap of FIG. 19 along line A-A.

FIG. 21 is an end view of the disinfecting cap of FIG. 17 showing an open end.

FIG. 22 is an end view of the disinfecting cap of FIG. 17 showing a closed end.

FIG. 23 is a perspective view of a disinfecting cap exemplary embodiment showing a closed end of the cap.

FIG. 24 is a perspective view of the disinfecting cap of FIG. 23 showing an open end of the cap.

FIG. 25 is a side view of the disinfecting cap of FIG. 23.

FIG. 26 is a cross-sectional view of the disinfecting cap of FIG. 25 along line A-A.

FIG. 27 is an end view of the disinfecting cap of FIG. 23 showing an open end.

FIG. 28 is an end view of the disinfecting cap of FIG. 23 showing a closed end.

FIG. 29 is a perspective, cross-sectional view of an alternative embodiment of the disinfecting cap of FIG. 26.

FIG. 30 is a perspective view of a disinfecting cap exemplary embodiment showing a closed end of the cap.

FIG. 31 is a perspective view of the disinfecting cap of FIG. 30 showing an open end of the cap.

FIG. 32 is a side view of the disinfecting cap of FIG. 30.

FIG. 33 is a cross-sectional view of the disinfecting cap of FIG. 32 along line A-A.

FIG. 34 is an end view of the disinfecting cap of FIG. 30 showing an open end.

FIG. 35 is an end view of the disinfecting cap of FIG. 30 showing a closed end.

FIG. 36 is a perspective view of a disinfecting cap exemplary embodiment showing an open end of the cap.

FIG. 37 is a perspective view of a disinfecting cap exemplary embodiment showing an open end of the cap.

FIG. 38 is a perspective view of a disinfecting cap exemplary embodiment showing an open end of the cap.

FIG. 39 is a perspective view of a skirt of the disinfecting cap of FIG. 38 showing a closed end of the skirt.

FIG. 40 is a perspective view of a skirt of the disinfecting cap of FIG. 38 showing an open end of the skirt.

FIG. 41 is a cross-sectional, perspective view of a skirt of the disinfecting cap of FIG. 38.

FIG. 42 is a perspective view of a cap body of the disinfecting cap of FIG. 38 showing an open end of the body.

FIG. 43 is a cross-sectional, perspective view of a cap body of the disinfecting cap of FIG. 38.

FIG. 44 is a cross-sectional view of a disinfecting cap exemplary embodiment.

FIG. 45 is a cross-sectional view of a disinfecting cap exemplary embodiment.

FIG. 46 is a perspective view of a disinfecting cap exemplary embodiment showing an open end of the cap.

FIG. 47 is a perspective, cross-sectional view of the disinfecting cap of FIG. 46.

FIG. 48 is a perspective view of a disinfecting cap exemplary embodiment showing an open end of the cap.

FIG. 49 is a perspective, cross-sectional view of the disinfecting cap of FIG. 48.

FIG. 50 is a perspective view of a disinfecting cap exemplary embodiment showing an open end of the cap.

FIG. 51 is a perspective, cross-sectional view of the disinfecting cap of FIG. 50.

FIG. 52 is a perspective view of a disinfecting cap exemplary embodiment showing the cap in an open configuration.

FIG. 53 is a perspective, cross-sectional view of a disinfecting cap exemplary embodiment.

FIG. 54 is a perspective, cross-sectional view of a cap body of the disinfecting cap of FIG. 53.

FIG. 55 is a perspective, cross-sectional view of a shell of the disinfecting cap of FIG. 53.

FIG. 56 is a perspective, cross-sectional view of a disinfecting cap exemplary embodiment.

FIG. 57 is a perspective, cross-sectional view of a cap body of the disinfecting cap of FIG. 56.

FIG. 58 is a perspective, cross-sectional view of a shell of the disinfecting cap of FIG. 56.

FIG. 59 is a perspective view of a disinfecting cap exemplary embodiment showing an open end of the cap.

FIG. 60 is a perspective, cross-sectional view of the disinfecting cap of FIG. 59.

FIG. 61 is a perspective view of a disinfecting cap exemplary embodiment showing an open end of the cap.

FIG. 62 is a perspective, cross-sectional view of the disinfecting cap of FIG. 61.

FIG. 63 is a perspective view of a disinfecting cap exemplary embodiment showing an open end of the cap.

FIG. 64 is a perspective, cross-sectional view of the disinfecting cap of FIG. 63.

FIG. 65 is a perspective view of a disinfecting cap exemplary embodiment showing an open end of the cap.

FIG. 66 is a perspective, cross-sectional view of the disinfecting cap of FIG. 65.

FIG. 67 is a perspective view of a disinfecting cap exemplary embodiment showing an open end of the cap.

FIG. 68 is a perspective, cross-sectional view of the disinfecting cap of FIG. 67.

FIG. 69 is a perspective, cross-sectional view of disinfecting cap exemplary embodiment.

FIG. 70 is a perspective view of the disinfecting cap of FIG. 69 showing an open end of the cap.

FIG. 71 is a perspective, cross-sectional view of the disinfecting cap of FIG. 69 showing a sponge positioned in the cap.

FIG. 72 is a perspective view of a sponge for use with various embodiments of a disinfecting cap.

FIG. 73 is a perspective view of a foil seal for use with various embodiments of a disinfecting cap.

FIG. 74 is a cross-sectional view of a Y-site having a female port.

FIG. 75 is a perspective view of a disinfecting cap exemplary embodiment showing an open end of the cap.

FIG. 76 is an elevation view of the disinfecting cap of FIG. 75 showing a side of the cap.

FIG. 77 is a cross-sectional view of the disinfecting cap of FIG. 75.

FIG. 78 is a perspective view of a disinfecting cap exemplary embodiment showing an open end of the cap.

FIG. 79 is an elevation view of the disinfecting cap of FIG. 78 showing a side of the cap.

FIG. 80 is a cross-sectional view of the disinfecting cap of FIG. 78.

FIG. 81 is a cross-sectional view of a disinfecting cap exemplary embodiment.

FIG. 82 is a cross-sectional view of a disinfecting cap exemplary embodiment.

FIG. 83 is an expanded cross-sectional view of section A of the disinfecting cap of FIG. 82.

FIG. 84 is a perspective view of a disinfecting cap exemplary embodiment showing an open end of the cap.

FIG. 85 is a cross-sectional view of the disinfecting cap of FIG. 84.

FIG. 86 is a perspective view of a disinfecting cap exemplary embodiment showing an open end of the cap.

FIG. 87 is a cross-sectional view of the disinfecting cap of FIG. 84.

FIG. 88 is a perspective, cross-section view of a disinfecting cap exemplary embodiment.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, forward and rearward, inside and outside, interior and exterior, and the like may be used solely to distinguish one entity or action from another entity or action without necessarily requiring or implying any actual such relationship, direction or order between such entities or actions.

The terms “proximal” and “distal” may be used in this application when describing various embodiments. These terms are not intended to be limiting and are merely provided for ease of maintaining a consistent orientation when describing various embodiments. As used herein, proximal refers to the direction generally closer to the patient and/or medical device to be cleaned and distal refers to the direction generally farther from the patient and/or medical device to be cleaned.

This application provides a description of various implementations and embodiments of a device for cleaning medical implements, in particular a device for cleaning vascular or other fluid access sites. Various embodiments of the invention include a cap having an opening to receive an access site. Throughout this application illustrative embodiments refer to use of a cap to engage with a “port” as an example of such an access site. One of skill in the art would understand that the invention may also be used in conjunction with other access sites or other medical devices without access sites.

In addition, the terms “open end” and “closed end” may be used in this application when describing various embodiments. These terms are not intended to be limiting and are merely provided for ease of maintaining a consistent orientation when describing various embodiments. As used herein, absent a specific contradictory disclosure, “open end” refers to an end of a cap having an opening into an interior volume into which a port is inserted, regardless of the geometry of the cap. Closed end refers to an end of a cap opposite or separated from the open end and not having an opening for accepting a port.

The following is a non-limiting example of how such a cap may be used by a healthcare worker: the healthcare worker may, with gloved hands, open the cap packaging and place the cap over the port of a medical implement to be cleaned. In certain embodiments, the healthcare worker may wipe the site by either applying a turning motion or by simply pushing the cap onto the port. The cap could then remain secured in place by threads other mechanisms described herein. A cap in place on a medical implement may be a positive indication that a desired site of the medical implement is clean. A vibrant color or other indicia may be used to allow instant visualization of a cap's presence from a door or hallway.

Embodiments of the cap described herein may include a disinfecting substance, such as a solution of a suitable microbiocide or germicide. The disinfecting substance can include an anti-bacterial disinfectant of any suitable type and suitable amount depending upon the size and structure of the cap. For example, in some embodiments the disinfecting substance may be an aqueous solution including about two percent (2%) chlorhexidine gluconate (chlorhexidine solution, “CHG”). In other embodiments, a solution including about 70 percent (70%) isopropyl alcohol (“IPA”) in an aqueous solution is included in the disinfecting substance. In yet another embodiment, a solution including about 70 percent (70%) IPA and about two percent (2%) CHG in an aqueous solution is included in the disinfecting substance. In the latter solution, it is recognized that the concentration of IPA can vary from about 60 percent (60%) to about 90 percent (90%) and the concentration of CHG can vary from about one percent (1%) to about five percent (5%), in one embodiment.

Other suitable solution compositions and concentrations are also possible. For instance, povidone iodine, polyhexanide (polyhexamethylene biguanide, “PHMB”), benzalkonium chloride (“BAC”), chlorxylenol (“PCMX”) or hydrogen peroxide solutions can be included in the disinfecting substance of further embodiments. Throughout this disclosure, reference to one or more of these disinfecting substances in relation to a cap embodiment should be understood to disclose the use of any other appropriate disinfecting substance as disclosed herein or as would be understood by one of ordinary skill in the art. In addition, embodiments of the disinfecting substance may be in a liquid or a gel form.

In various embodiments of the invention described herein, the port comes in contact with the disinfecting substance in liquid or gel form, or with an absorbent material infused with the disinfecting substance. However, it may not be necessary for the surface of the port to contact the liquid or gel disinfecting substance. For example, if IPA is used as part of a disinfecting solution, IPA vapors trapped within the cap may act as a disinfectant for the port without requiring contact between the liquid solution and the site being cleaned.

Various materials may be used to manufacture the cap embodiments described herein. Appropriate materials may include polyurethane (“PU”), polypropylene (“PP”), polyethylene (“PE”), thermoplastic elastomer (“TPE”), thermoplastic vulcanizate (“TPV”) such as Santoprene, or other materials as would be understood by one of ordinary skill in the art. Various embodiments described herein recite the use of particular materials, but one of ordinary skill would understand that other appropriate materials could be substituted for the disclosed material.

Various embodiments also describe the use of sponge or foam material. Such foam may be formed of polyurethane (“PU”) or another appropriate absorbent material. Alternatively, other absorbent materials may be used in place of foam, including for example, felted non-woven or other fibrous materials.

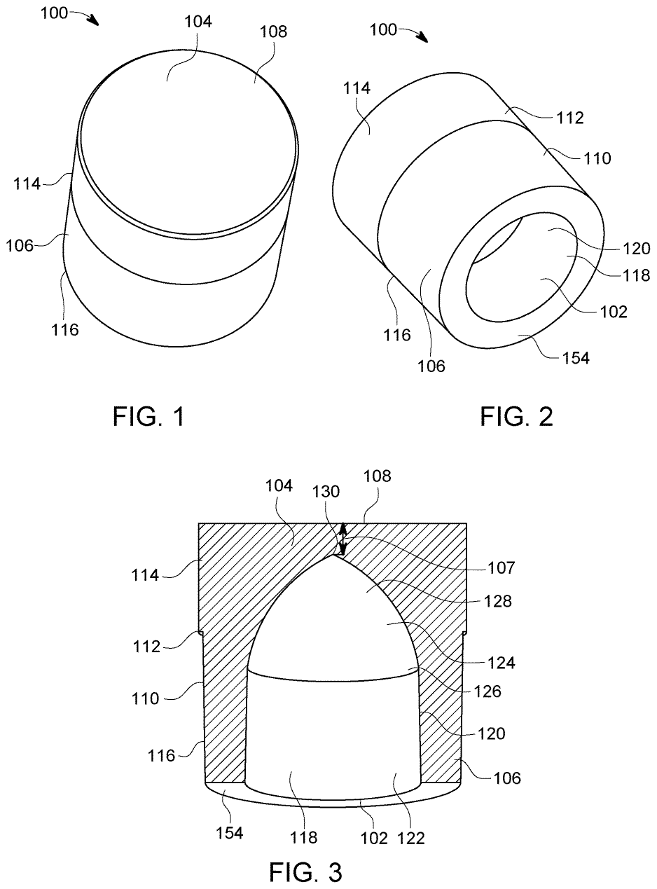

FIGS. 1-3 show an illustrative embodiment of a disinfecting cap. The cap 100 may comprise an open end 102 and a closed end 104. A sidewall 106 may extend between the open end and closed end. An end wall 108 may be positioned at the closed end 104 of the cap. An outside surface 110 of the sidewall 106 may be generally cylindrical and may comprise a step 112 such that a first portion 114 of the sidewall has a diameter that is greater a second portion 116 of the sidewall. Alternatively, the first and second portions may have substantially the same diameter, or the first portion of the sidewall may have a diameter that is less than the diameter of the second portion.

The sidewall 106 may encompass an interior volume 118. An interior surface 120 of the sidewall 106 may be generally cylindrical in a first section 122 of the interior volume. Alternatively, the interior volume may have a generally conical cross-section in the first section 122. The interior volume may also comprise a second section 124. The second section may comprise an open end 126 and a closed end 128. A cross-section of the second section 124 may be spherical, semi-spherical, conical, a rounded cone, or some combination of these or other shapes. A distance 107 between the closed end 128 of the second section and the end wall 108 may vary depending on the dimensions of the cap and shape of the second section. A post 130 may extend into the interior space 118 from the closed end 128 of the second section 124.

The disinfection cap 100 may comprise a single material that forms the sidewall 106 and end wall 104 in a single piece. For example, the disinfecting cap 100 may comprise a unitarily molded piece. The cap may be formed of TPV or any other appropriate material.

The cap may further comprise a cover or seal as illustrated in FIG. 73 that extends across the open end 102 of the cap to seal a disinfecting substance and/or an absorbent material within the interior volume. The seal may be positioned adjacent to an end surface 154 of the interior volume 118 or may be spaced some distance into the interior volume, leaving a space between the seal and the end surface. The seal may have a first portion 408 generally shaped and sized to cover the open end 102 of the cap. The seal may further include a grip or tab 410 that aids in removal of the seal from the cap. Alternatively, the seal may include perforations, score lines or other features to aid in fracturing the seal. Such a seal may be any appropriate material including foil, polymer, etc.

The interior surface 120 of the first section 122 may engage an outside surface of a female luer 6802, as shown, for example, in the Y-site 6800 illustrated in FIG. 74. The interior surface 120 of the cap may engage the threads 6804 of the female luer. The engagement may be made by a press or friction fit between an outside edge 6806 of the threads against the cap interior surface 120. In such a press fit, the interior surface of the cap may deform outwardly to accommodate the threads of the female luer. Such deformation may occur by a displacement of some or all the cap sidewall 106 in an outward direction. Alternatively, deformation of the interior surface 120 may occur by compression of the sidewall material without movement or deformation of the outside surface 110. In further alternatives, both compression and movement of the sidewall may occur on inserting the female luer into the cap. Embodiments of the disinfecting cap may incorporate elements similar to those discussed elsewhere in the application but not specifically labeled in or discussed with respect to FIGS. 1-3.

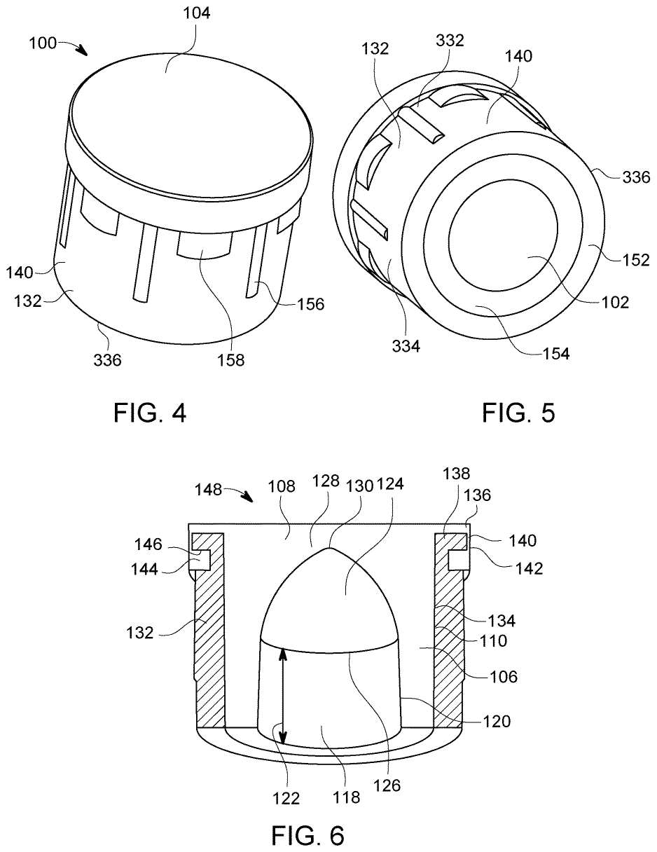

FIGS. 4-6 show a further illustrative embodiment of a disinfecting cap. The cap 100 may comprise an open end 102 and a closed end 104. A sidewall 106 may extend between the open end and closed end. An end wall 108 may be positioned at the closed end 104 of the cap. An outside surface 110 of the sidewall 106 may be generally cylindrical.

The sidewall 106 may encompass an interior volume 118. An interior surface 120 of the sidewall 106 may be generally cylindrical in a first section 122 of the interior volume. Alternatively, the interior volume may have a generally conical cross-section in the first section 122. The interior volume may also comprise a second section 124. The second section may comprise an open end 126 and a closed end 128. A cross-section of the second section 124 may be spherical, conical, a rounded cone, or some combination of these or other shapes. A post 130 may extend into the interior space 118 from the closed end 128 of the second section 124.

The disinfection cap 100 may comprise a skirt 132 that surrounds a least a portion of the sidewall 106. An inside surface 134 of the skirt 132 may be positioned adjacent to the outside surface 110 of the sidewall 106. The inside surface of the skirt may be adhered to the outside surface of the sidewall. A portion 136 of the sidewall 106 may extend around an end 138 of the skirt 132. The flange 142 of the sidewall may extend to cover an outside surface 140 of the skirt. An extension 144 of the flange 142 may engage with a notch or channel 146 formed in an outer surface 140 of the skirt. The notch 146 may extend around a portion or substantially the entire circumference of the skirt. The flange 142 and notch 146 may be formed adjacent or near the closed end 104 of the cap. Alternatively, one or more tabs may extend from the skirt 132 or sidewall flange 142 that engage with corresponding indentations or the like that are formed in the corresponding surface of the sidewall extension or skirt. The skirt 132 may extend along the sidewall 106 of the cap to the open end 102. An open-end surface 152 of the skirt may be coplanar with an end surface 154 of a cap body 148 at an open end 102 of the cap. In this manner, the skirt end surface 152 and cap body end surface 154 may form concentric rings at an open end of the cap.

The cap body 148 of the cap may be formed of a first material. This material may be TPV, PU, or another appropriate material. The skirt 132 may be formed of a second material. In embodiments of the cap, the second material has a higher durometer than the first material. The second material may be PP, PE, or another appropriate material.

The interior surface 120 of the first section 122 may engage an outside surface of a female luer 6802, as shown, for example, in the Y-site 6800 illustrated in FIG. 74. The interior surface 120 of the cap may engage the threads 6804 of the female luer. The engagement may be made by a press or friction fit between an outside edge 6806 of the threads against the cap interior surface 120. In such a press fit, the interior surface of the cap may deform outwardly to accommodate the threads of the female luer. Such deformation may occur by a displacement of some or all the cap sidewall 106 in an outward direction. Alternatively, deformation of the interior surface 120 may occur by compression of the sidewall material without movement or deformation of the outside surface 110. In further alternatives, both compression and movement of the sidewall may occur on inserting the female luer into the cap.

The skirt 132 may constrain or provide reinforcement to the cap body 148. In embodiments of the cap, the skirt may reduce or eliminate any expansion of the cap when a female luer is inserted; the skirt may increase the pressure exerted by the cap against the luer, which may result in an increased force necessary to remove the cap once it is positioned on the luer; or the skirt may have both these effects. An outer surface 140 of the skirt may comprise protuberances that extend from the outer surface such as columns 156 or tabs 158 as described in more detail elsewhere in the application. Embodiments of the disinfecting cap may incorporate elements similar to those discussed elsewhere in the application but not specifically labeled in or discussed with respect to FIGS. 4-6.

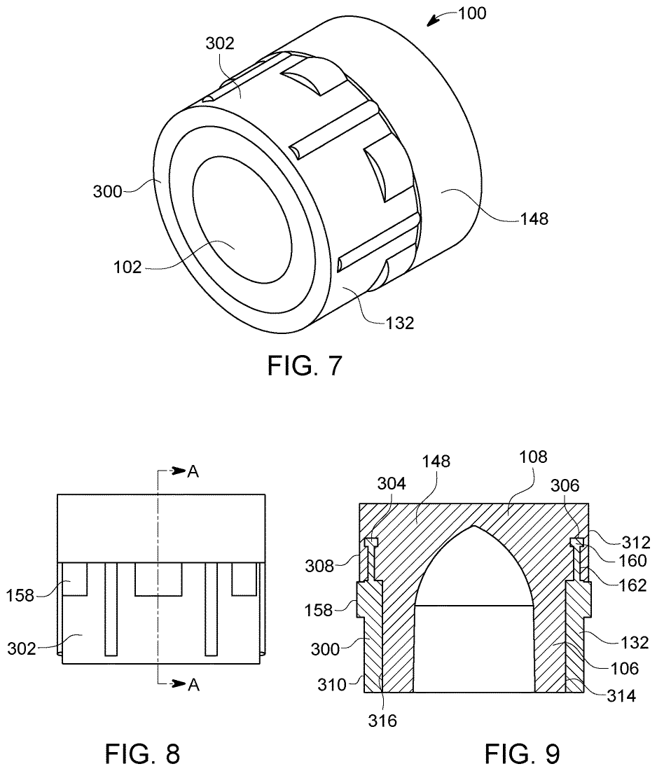

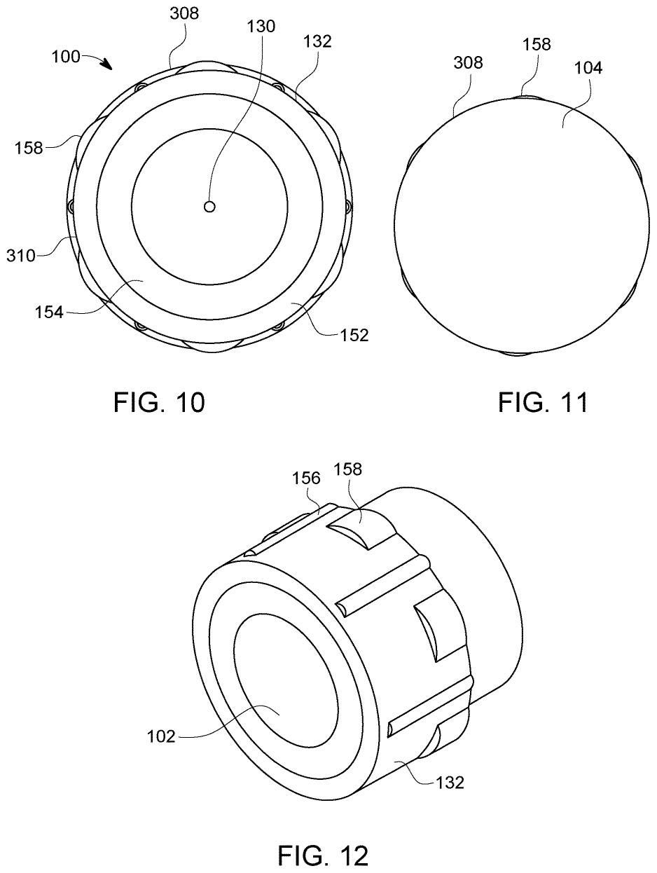

FIGS. 7-11 show a further illustrative embodiment of a disinfecting cap. The cap 100 may comprise a cap body 148 having an open end 102 and a closed end 104. A sidewall 106 may extend between the open end and closed end. An end wall 108 may be positioned at the closed end 104 of the cap. The disinfection cap 100 may comprise a skirt 132 that surrounds a least a portion of the sidewall 106. A skirt flange 160 may interlock or otherwise engage with a groove 162 of the cap body 148. The skirt flange 160 may have a cross section that is narrower than the cross section of skirt wall 300 of the skirt body 302. The flange may comprise a stepped engagement ring 304 or discrete tabs that extend outward from the flange sidewall. The engagement ring 304 may engage a corresponding cavity 306 formed in the cap body groove 162 and may comprise a tab or extension 305. Alternatively, the cap may comprise other engagement elements formed on the skirt flange 160 that engage with corresponding elements formed in the body groove 162, including tabs, serrations, threads, or the like.

In embodiments of the cap, the cap body may have a first section 312 having a first diameter, and second section 314 having a second diameter that engages an inner diameter/surface 316 of the skirt. The inner surface of the skirt may be generally cylindrical or may be in the shape of a truncated cone with the open end 384 of the surface 316 having a smaller diameter than the closed end 386. A flange 142 of the cap body 148 may extend beyond an outer surface 310 of the skirt body 302. Tabs 158 may extend from the skirt outer surface 310 beyond the outer surface 308 of the cap body 148. Embodiments of the disinfecting cap may incorporate elements similar to those discussed above but not specifically labeled in or discussed with respect to FIGS. 7-11.

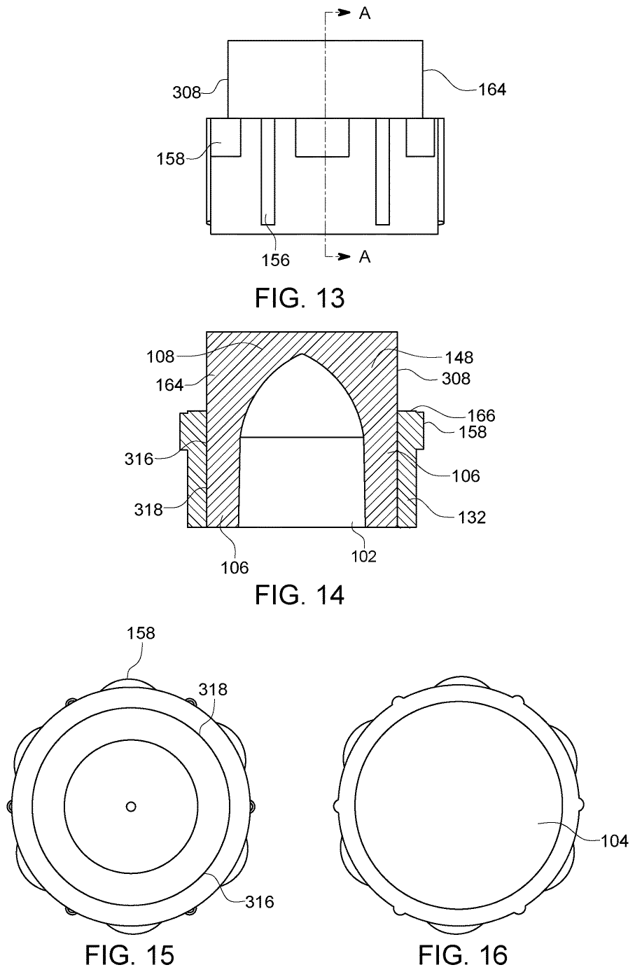

FIGS. 12-16 show a further illustrative embodiment of a disinfecting cap. The cap 100 may comprise a cap body 148 having an open end 102 and a closed end 104. A sidewall 106 may extend between the open end and closed end. An end wall 108 may be positioned at the closed end 104 of the cap. The disinfection cap 100 may comprise a skirt 132 that surrounds a least a portion of the sidewall 106. An outer surface 308 of the of the cap body sidewall may have a uniform, cylindrical outside diameter 318 that engages an inner diameter 316 of the skirt. A section 164 of the cap body sidewall 106 may extend beyond a bottom edge 166 of the skirt. Embodiments of the disinfecting cap may incorporate elements similar to those discussed elsewhere in the application but not specifically labeled in or discussed with respect to FIGS. 12-16.

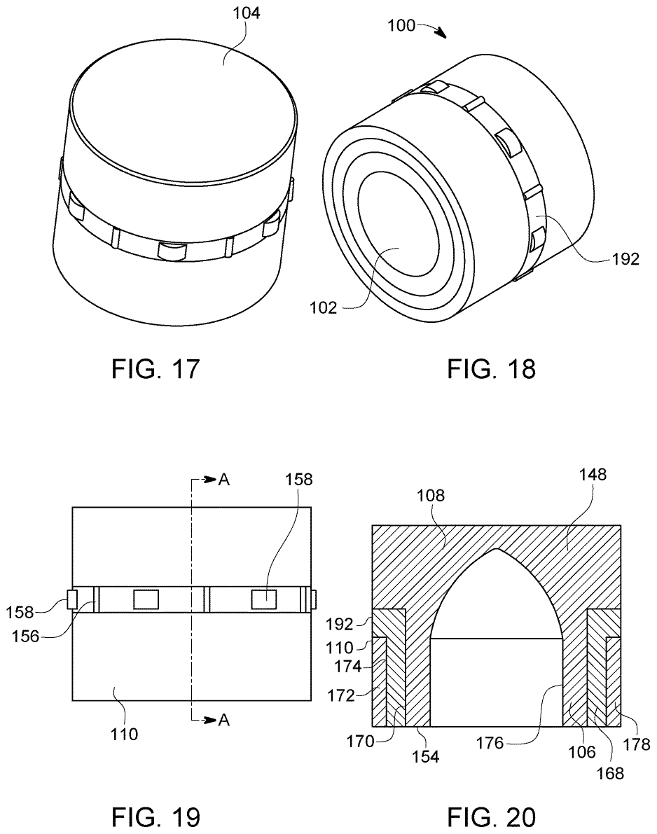

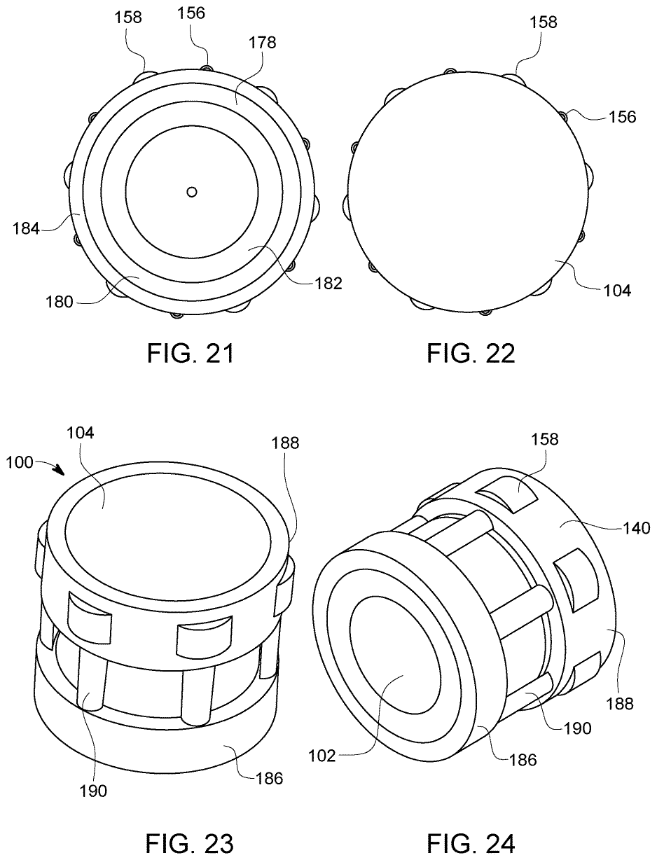

FIGS. 17-22 show a further illustrative embodiment of a disinfecting cap. The cap 100 may comprise a cap body 148 having an open end 102 and a closed end 104. A sidewall 106 may extend between the open end and closed end. An end wall 108 may be positioned at the closed end 104 of the cap. The disinfection cap 100 may comprise a framework 168 that is positioned within or embedded in the sidewall 106 such that a part 176 of the sidewall is inside the framework and a part 178 of the sidewall is outside the framework. Alternatively, the framework may be positioned around an outer surface 170 of the sidewall 106, and a secondary sidewall 172 may surround a framework outer surface 174. The framework may include voids or through holes (not shown) that are filled by the material of the cap body 148 allowing the framework 168 to interlock with the cap body. An open-end surface 180 of the framework 168 may be coplanar with an end surface 154 of a cap body 148 at an open end 102 of the cap. In this manner, the framework end surface 180, the cap body inside part end surface 182, and the cap body outside part end surface 184 may form concentric rings at an open end of the cap. A section 192 of the framework may be concentric with or otherwise exposed at an outside surface 110 of the sidewall 106. Columns 156 and/or tabs 158 may extend from the exposed section 192. Embodiments of the disinfecting cap may incorporate elements similar to those discussed elsewhere in the application but not specifically labeled in or discussed with respect to FIGS. 17-22.

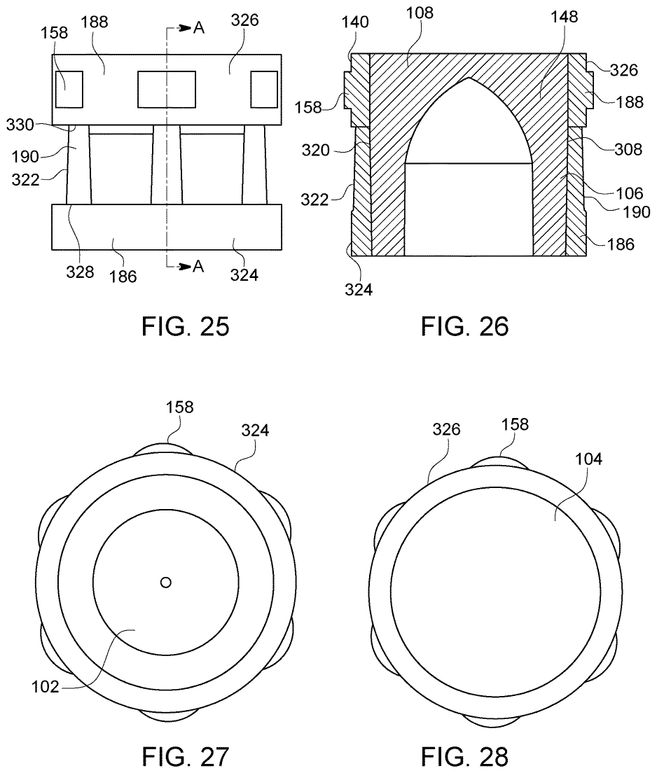

FIGS. 23-28 show a further illustrative embodiment of a disinfecting cap. The cap 100 may comprise a cap body 148 having an open end 102 and a closed end 104. A sidewall 106 may extend between the open end and closed end. An end wall 108 may be positioned at the closed end 104 of the cap. The disinfection cap 100 may comprise a first ring 186 that surrounds a least a portion of the sidewall 106. The first ring may be positioned near or adjacent the open end 102 of the cap. The cap may further comprise a second ring 188 that surrounds a least a portion of the sidewall 106. The second ring may be positioned adjacent the closed end 102 of the cap or nearer to the closed end relative to the first ring. Columns 190 may extend between the first ring 186 and the second ring 188. Tabs 158 may extend from an outer surface 140 of one or both rings. The columns may comprise an outer surface 322 having a generally semi-circular cross-section and an inner surface 320 that conforms to the outer surface 308 of the cap body. The column outer surface 322 may be recessed such that an outer surface 324 of the first ring 186 and an outer surface 326 of the second ring 188 extend beyond the surface 322 of the columns. The columns 190 may have a tapered cross section such that the column has a greater cross-sectional area at a first end 328 than at a second end 330. The first and second rings may have the same outside diameter. Alternatively, the first and second rings may have different outside diameters. Embodiments of the disinfecting cap may incorporate elements similar to those discussed elsewhere in the application but not specifically labeled in or discussed with respect to FIGS. 23-28.

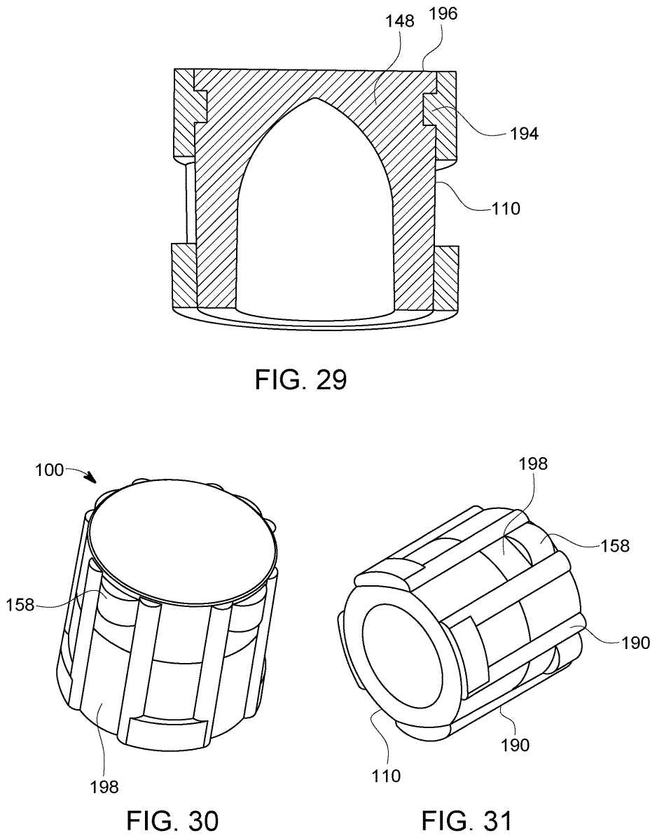

FIG. 29 show a further illustrative embodiment of the disinfecting cap of FIGS. 23-28. In this embodiment, one or more of the rings 186, 188 may comprise an inwardly extending flange 194. The flange may engage a groove 196 formed in an outside surface 110 of the cap body 148.

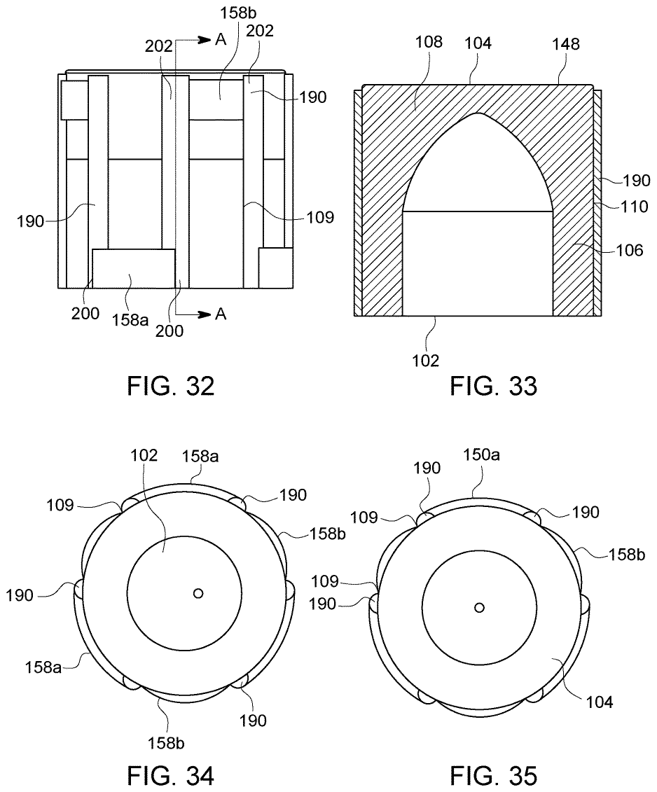

FIGS. 30-35 show a further illustrative embodiment of a disinfecting cap. The cap 100 may comprise a cap body 148 having an open end 102 and a closed end 104. A sidewall 106 may extend between the open end and closed end. An end wall 108 may be positioned at the closed end 104 of the cap. The disinfection cap 100 may comprise columns 190 extending along and from an outer surface 110 of the cap body. Tabs 158 may extend along or from the outer surface 110 of the cap body 148 in a space 198 between the columns 190. The tabs 158 may attach adjacent first ends 200 or adjacent second ends 202 of the columns 190. Tabs may alternate between attaching first ends 200 and second ends 202. Tabs 158a may extend from a centerline of adjacent columns 190. Alternatively, tabs 158b may extend from an edge 159 or near to an edge of adjacent columns 190. Both tab configurations 158a, 158b may be used. For example, longer tabs 158a may be used adjacent to the open end 102 of the cap body 148, and shorter tabs 158b may be used adjacent to the closed end 104 of the cap body. Embodiments of the disinfecting cap may incorporate elements similar to those discussed elsewhere in the application but not specifically labeled in or discussed with respect to FIGS. 30-35.

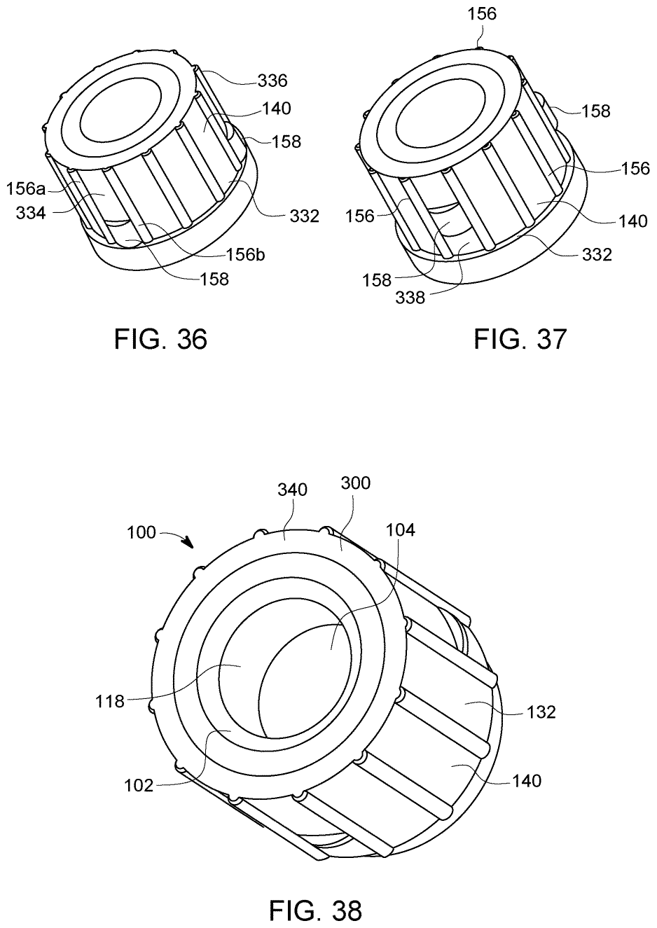

As particularly illustrated in FIGS. 36 and 37 and as shown in other figures, an outer surface 140 of the skirt may comprise protuberances that extend from the outer surface such as columns 156 or tabs 158. The columns 156 may be of various lengths, heights, and thicknesses and may vary in number and positioning. The columns 156 may extend a full length of the exposed outside surface 334 of the skirt from a lower edge 332 of the exposed section to an upper edge 336. Alternatively (see, e.g., FIGS. 4-6), the columns may extend from a lower edge 332 of the exposed section 334 of the skirt for a distance that does not extend to the upper edge 336. The columns may provide for an enhanced grip by the user to improve control and manipulation of the cap. The tabs 158 may be of various lengths and heights and may vary in number and positioning. For example, the tabs may be positioned adjacent to the lower edge 332 of the skirt exposed section 334. Alternatively, the tabs may be spaced apart some distance 338 from the lower edge 332. A tab 158 may extend from one column 156a to an adjacent column 156b. Alternatively, the tab may extend a distance less than the space between columns (see, e.g., FIGS. 4-6). The tabs may allow the cap to engage with other devices. For example, if the cap is positioned in a cavity of a syringe plunger, the tabs 158 may interlock with features of the plunger cavity to secure the cap in position until the cap is to be used on a site.

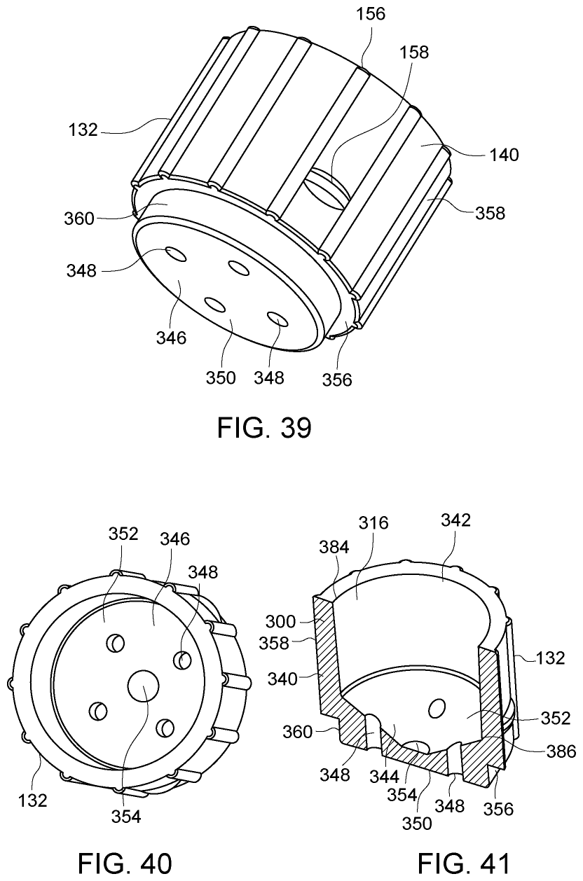

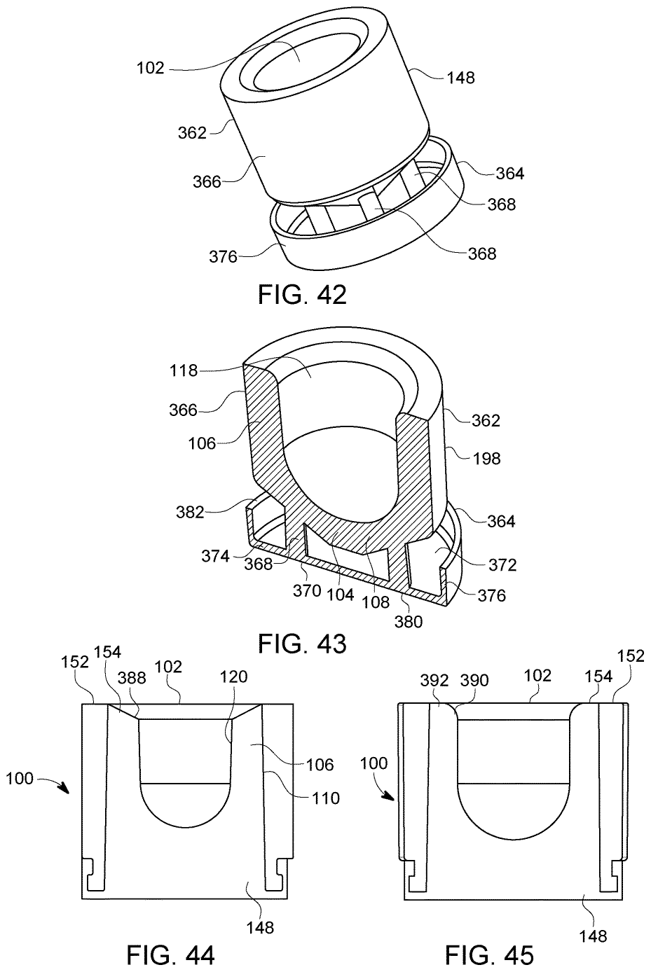

FIGS. 38-43 show a further illustrative embodiment of a disinfecting cap. The cap 100 may comprise a cap body 148 having an open end 102 and a closed end 104. A sidewall 106 may extend between the open end and closed end. An end wall 108 may be positioned at the closed end 104 of the cap. The disinfection cap 100 may comprise a skirt 132 that surrounds a least a portion of the sidewall 106. The skirt 132 may comprise a skirt side wall 340 and a skirt end wall 346. The skirt may further comprise an outer surface 140. The outer surface 140 of the skirt may comprise protuberances that extend from the outer surface such as columns 156 or tabs 158 as described in more detail elsewhere in the application. The skirt may further comprise an open end 342 and a closed end 344. The closed end may have an end wall 346. Holes, channels, or apertures 348 may extend through the end wall 346. The end wall 346 may be generally planar, while an upper or inside surface 352 of the end wall may have a convex shape. The convex shape may be at least partially spherical or may be formed in a truncated cone shape. The inside surface 352 may have a generally planar portion 354 at a center of the inside surface. The skirt outer surface 140 may comprise a lower surface 350 of the end wall and may further comprise a step 356 between a first, upper section 358 having a first diameter and a base section 360 having different diameter. The diameter of the base section may be smaller than the diameter of the upper section.

The cap body 148 may comprise a cavity portion 362 and a flange portion 364. The cavity portion may comprise a cavity or interior volume 118 extending from the open end 102. The cavity portion may have a sidewall 106 that is surrounded, at least in part, by wall 300 of the skirt 132 such that an outside surface 366 of the cavity portion sidewall 106 is adjacent to an inner diameter 316 of the skirt sidewall 340. Pillars or extensions 368 may extend from an outer bottom surface 370 or the cavity portion 362. The pillars 368 extend through channels 348 formed in the skirt end wall 346.

The pillars 368 may connect the cavity portion 362 with the flange portion 364. The flange portion may comprise a bottom or end wall 380 having an inner surface 372, an outer surface 374, and a sidewall 376 extending from an edge 378 of the bottom wall in a direction toward the open end of the cavity portion. The sidewall 376 may be spaced and sized such that it surrounds the base section 360 of the skirt 132. An end surface 382 of the sidewall 376 may contact or be positioned proximate the skirt step 356. The pillars 368 may extent from the base section 360 inner surface 372 through the channels in the skirt end wall 346. Embodiments of the disinfecting cap may incorporate elements similar to those discussed above but not specifically labeled in or discussed with respect to FIGS. 38-43.

The cap body cavity portion 362 and flange portion may be integrally formed. In an illustrative method of manufacturing, the skirt 132 may be injection molded from an appropriate material as discussed elsewhere in the application. The cap body 148 may then be overmolded by injection molding onto the skirt in a secondary operation. The channels 348 through the skirt allow the cap body portions to be molded in a single piece and provide for an interlocking engagement between the cap body and the skirt.

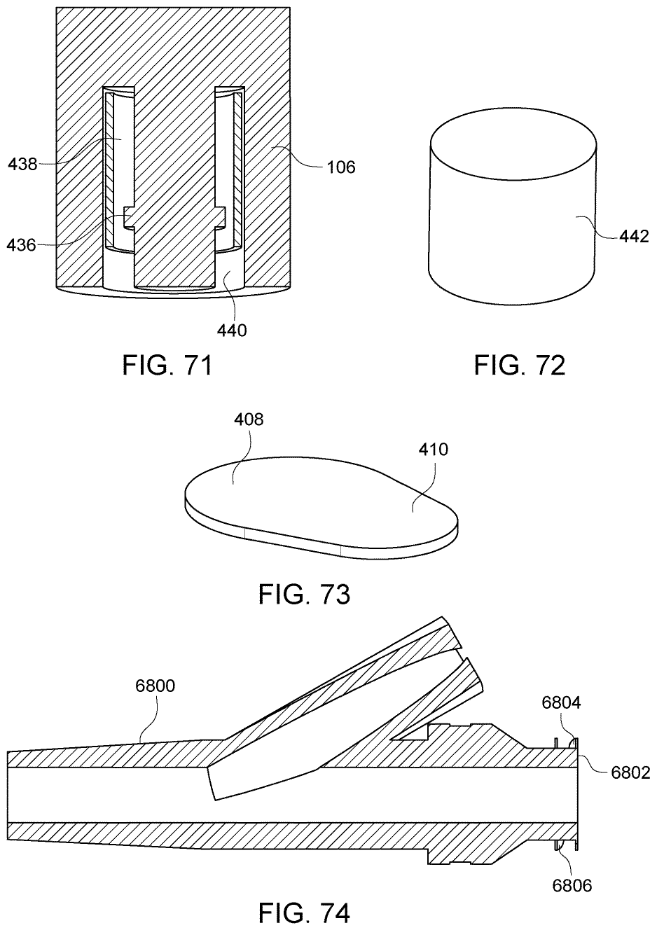

FIGS. 44-71 illustrate additional embodiments or features of a disinfecting cap. These features and embodiments may incorporate elements or features of other cap embodiments discussed herein or may be incorporated into such other embodiments.

As illustrated in FIGS. 44-45, an open end surface 152 of the skirt may be coplanar with an end surface 154 of a cap body 148 at an open end 102 of the cap 100. The cap body may provide for a transition from the interior surface 120 of the interior volume 118 to the end surface 154 of the cap body. The transition may comprise a chamfer 388 (FIG. 44). The chamfer 388 may extend from the interior surface 120 to the outside surface 110 of the sidewall 106 such that the chamfer forms the entire end surface 154 of the cap body. Alternatively, the chamfer may extend across only a portion of the end surface such that a section of the end surface is coplanar with the end surface 152 of the skirt. The transition may comprise a fillet 390 (FIG. 45). The fillet may be sized such that a section 392 of the end surface is coplanar with the end surface 152 of the skirt. Alternatively, the fillet may be sized such that the fillet extends across substantially the entire end surface 154 of the cap body.

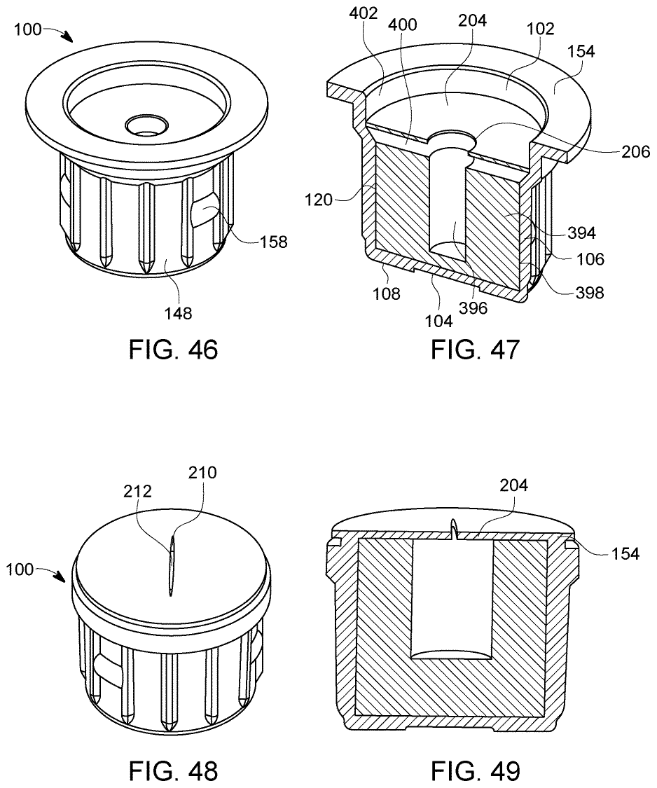

FIGS. 46-47 show a further illustrative embodiment of a disinfecting cap. The cap 100 may comprise a cap body 148 having an open end 102 and a closed end 104. A sidewall 106 may extend between the open end and closed end. An end wall 108 may be positioned at the closed end 104 of the cap. The cap may comprise a membrane 204 that extends at least partial across the opening formed at the open end 102 of the cap. The membrane may comprise a hole 206 formed at its center. The membrane may comprise an elastomeric material that allows displacement of the membrane so that a site can be inserted into the cap. The membrane may be spaced some distance 402 below an end surface 154 of the cap.

The cap may further comprise a sponge 394 or other absorbent material that is placed in the interior 118 of the cap. The sponge may comprise a central cavity 396. The sponge may be cylindrical with an outside diameter such that an outside surface 398 of the sponge is proximate an interior surface 120 of the cap. The central cavity may extend from a top surface 400 of the sponge for a distance that is less than the full thickness of the sponge. Alternatively, the central cavity may extend through the entire thickness of the sponge.

FIGS. 48-49 show further embodiments of a disinfecting cap. The cap 100 may comprise a membrane 204 that extends at least partial across the opening formed at the open end 102 of the cap. The membrane may comprise a slit 210 formed at or extending over a center point 212 of the membrane. The membrane may be positioned such that the membrane is coplanar with the end surface 154 of the cap.

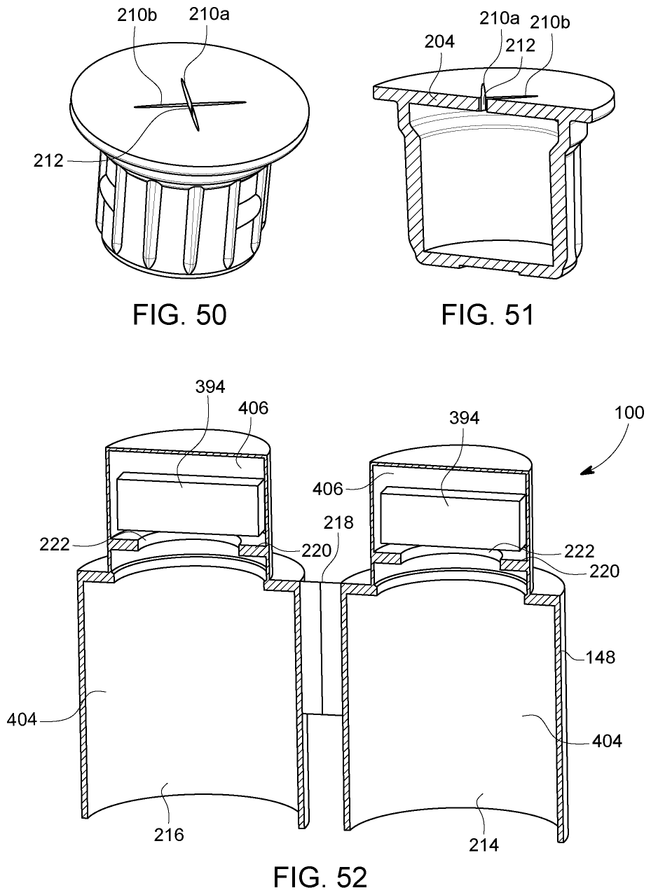

FIGS. 50-51 show further embodiments of a disinfecting cap. The cap 100 may comprise a membrane 204 that extends at least partial across the opening formed at the open end 102 of the cap. The membrane may comprise multiple slits 210a, 210b. One or more of the slits may extend over a center point 212 of the membrane. The slits may intersect at the center point. The slits may extend at a 90° angle relative to one another within the plane of the membrane. The membrane may comprise an elastomeric material that allows displacement of the membrane so that a site can be inserted into the cap.

FIG. 52 show a further illustrative embodiment of a disinfecting cap. The cap 100 may comprise a cap body 148. The cap body may include a first section 214 and a second section 216. A hinge 218 connects the first and second body sections 214, 216 such that the cap may be snapped around a site. Each body section may contain a portion of a membrane 220. The membrane portions may include cut-outs 222. The membrane may separate a first cavity 404 of the cap 100 from a second cavity 406. One or more sponges 394 may be positioned in the second cavity. The cap may comprise a latch or other engaging elements that secure the cap in a closed position.

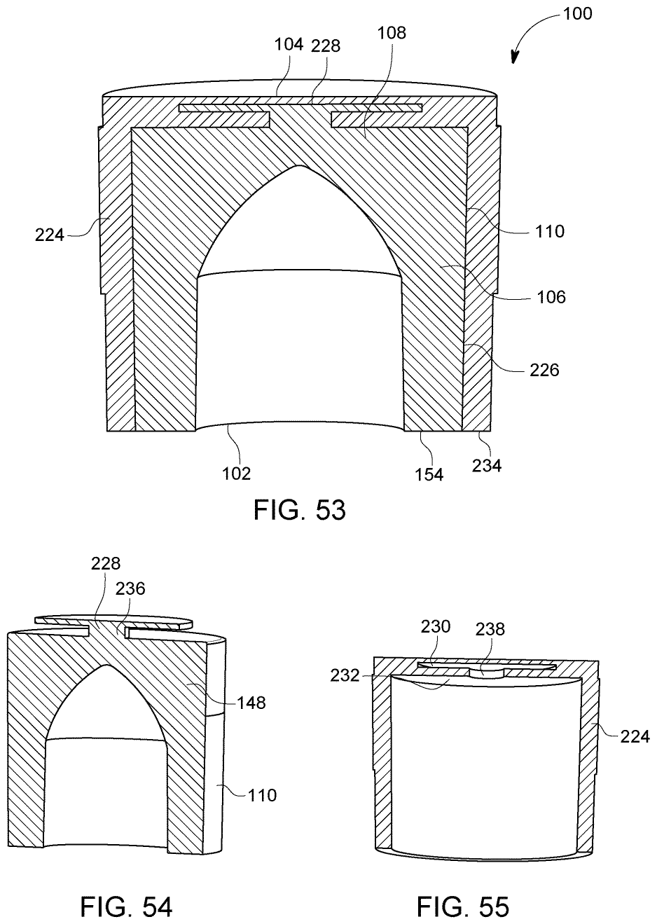

FIGS. 53-55 show a further illustrative embodiment of a disinfecting cap. The cap 100 may comprise an open end 102 and a closed end 104. A sidewall 106 may extend between the open end and closed end. An end wall 108 may be positioned at the closed end 104 of the cap. An outside surface 110 of the sidewall 106 may be generally cylindrical.

The disinfection cap 100 may comprise a shell 224 that surrounds a least a portion of the sidewall 106 and the end wall 108. An inside surface 226 of the shell 224 may be positioned adjacent to the outside surface 110 of the sidewall 106. The inside surface of the shell may be adhered to the outside surface of the sidewall. A flange 228 may extend from an outside surface of the cap body end wall 108. The flange 228 may engage a groove 230 formed at an inside surface 232 of the shell 224. The flange 228 may be spaced from the cap body end wall 108 by a post section 236 that corresponds to a hole 238 formed in the shell inside surface 232.

An open-end surface 234 of the shell may be coplanar with an end surface 154 of a cap body 148 at an open end 102 of the cap. In this manner, the shell end surface 234 and cap body end surface 154 may form concentric rings at an open end of the cap.

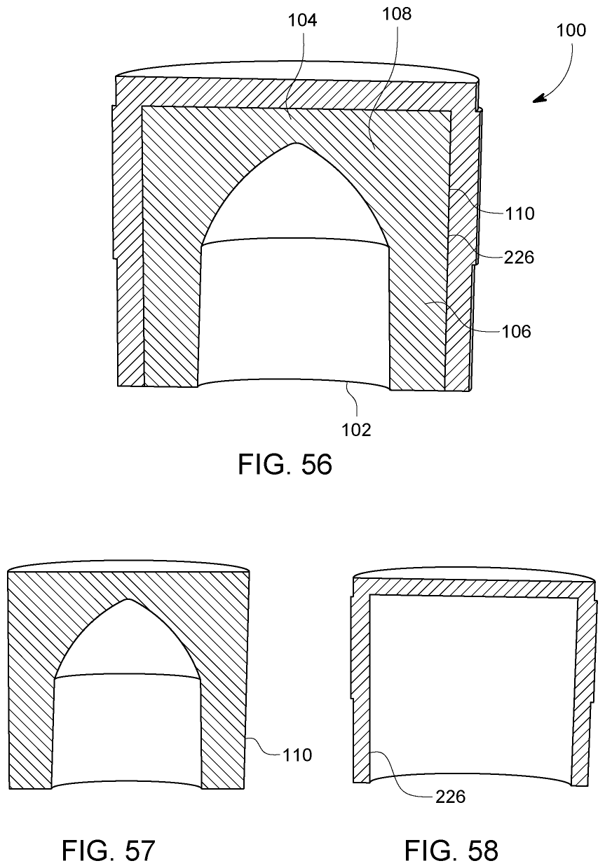

FIGS. 56-58 show a further illustrative embodiment of a disinfecting cap. The cap 100 may comprise an open end 102 and a closed end 104. A sidewall 106 may extend between the open end and closed end. An end wall 108 may be positioned at the closed end 104 of the cap. An outside surface 110 of the sidewall 106 may be generally cylindrical.

The disinfection cap 100 may comprise a shell 224 that surrounds a least a portion of the sidewall 106 and the end wall 108. An inside surface 226 of the shell 224 may be positioned adjacent to the outside surface 110 of the sidewall 106. The inside surface of the shell may be adhered to the outside surface of the sidewall.

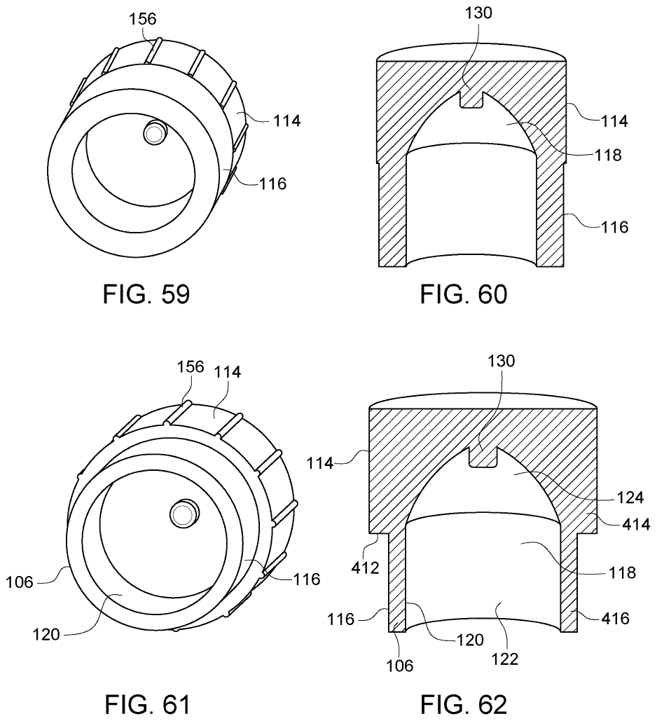

FIGS. 59-71 illustrate additional configuration of embodiments of the cap as discussed throughout the application. These configurations disclose different features of the embodiments, and caps consistent with these configurations may have a unitary construction as discussed with regard to the embodiment of FIGS. 1-3 or may have a two-part construction as discussed with regard to the embodiment of FIGS. 4-6 and others.

FIGS. 59-60 illustrate an embodiment in which columns 156 extend from a first portion 114 of the sidewall but do not extend to a second portion 116. A post 130 may extend into the interior space 118. Apart from the columns 156, the first 114 and second 116 portions have substantially the same cross section. Other features of the embodiment will be apparent from the figures.

FIGS. 61-62 illustrate an embodiment in which columns 156 extend from a first portion 114 of the sidewall but do not extend to a second portion 116. A post 130 may extend into the interior space 118. The first 114 portion has a diameter that is larger than the second section such that a step 412 is formed between the sections. The sidewall 414 of the first portion is thicker than the sidewall 416 of the second portion 116. An interior surface 120 of the sidewall 106 may be generally cylindrical in a first section 122 of the interior volume. The interior volume may also comprise a second section 124. A cross-section of the second section 124 may have a rounded conical shape. Other features of the embodiment will be apparent from the figures.

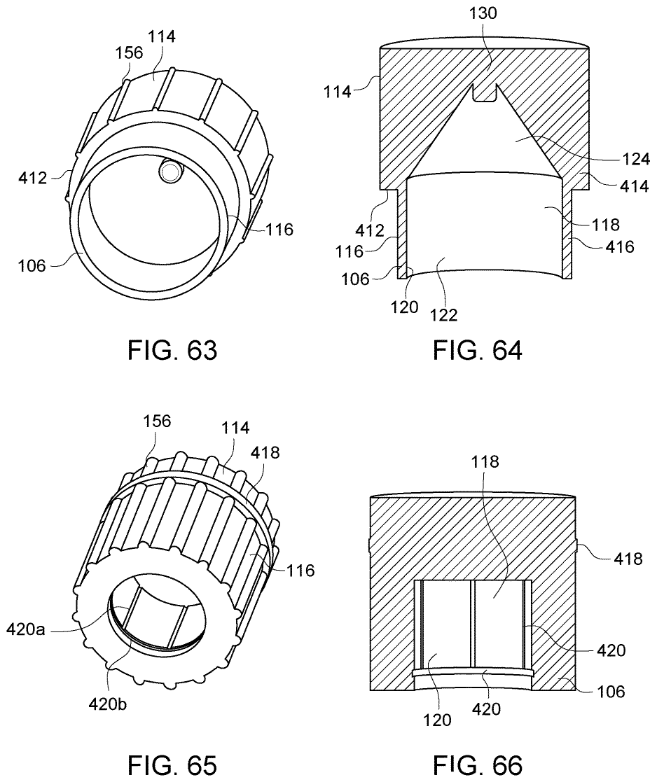

FIGS. 63-64 illustrate an embodiment in which columns 156 extend from a first portion 114 of the sidewall but do not extend to a second portion 116. A post 130 may extend into the interior space 118. The first 114 portion has a diameter that is larger than the second section such that a step 412 is formed between the sections. The sidewall 414 of the first portion is thicker than the sidewall 416 of the second portion 116. An interior surface 120 of the sidewall 106 may be generally cylindrical in a first section 122 of the interior volume. The interior volume may also comprise a second section 124. A cross-section of the second section 124 may have a conical shape. Other features of the embodiment will be apparent from the figures.

FIGS. 65-66 illustrate an embodiment in which columns 156 extend from a first portion 114 of the sidewall and a second portion 116. A raised ring or divider 418 may extend from the outer surface of the cap between the first 114 and second 116 portions. The divider 418 may have a diameter that extends beyond the surface of columns 156. Alternatively, the divider may have a diameter that is coextensive with the highest point of the columns. An interior surface 120 of the sidewall 106 may be generally cylindrical. The interior surface 120 may comprise channels or grooves 420 formed in the surface. A groove 420b may extend concentrically around the interior volume 118 and/or a groove 420a may extend longitudinally along the interior surface. Other features of the embodiment will be apparent from the figures.

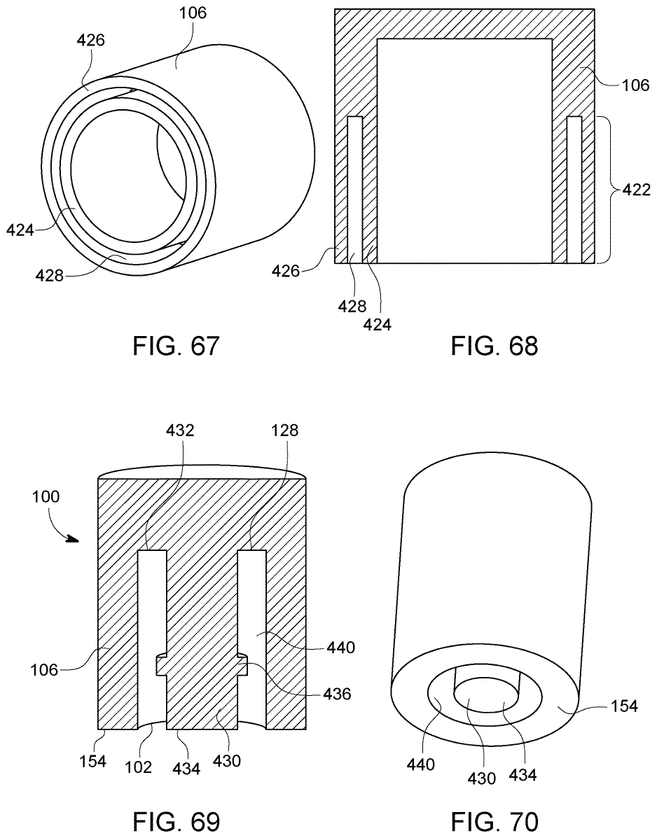

FIGS. 67-68 illustrate an embodiment in which a portion 422 the cap sidewall 106 comprises an interior section 424 and an exterior section 426 that are separated by an annular cavity 428. The interior section 424 of the sidewall may flex outwardly when a site is inserted into the cap in order to accommodate and secure the site. Other features of the embodiment will be apparent from the figures.

FIGS. 69-71 illustrate an embodiment in which a central post 430 extends from the center of a bottom surface 432 at the closed end 128 of the cap 100. The post may extend from the bottom surface to the open end 102 of the cap such that a top surface 434 of the post is coplanar with an end surface 154 of the side wall 106, thereby forming and annular interior space 440. A ring or tab protrusions 436 may extend from a side surface 438 of the post. The protrusions 436 may be positioned between the interior space bottom surface 432 and the post top surface 434. An annular sponge 438 may be positioned in the annular interior space 440. Other features of the embodiment will be apparent from the figures.

FIG. 72 illustrates an embodiment of a foam material or sponge 442 that may be positioned within the interior volume 118 of various embodiments of the cap 100. The sponge may be generally cylindrical as shown or, alternatively, cubical, spherical, or another appropriate shape.

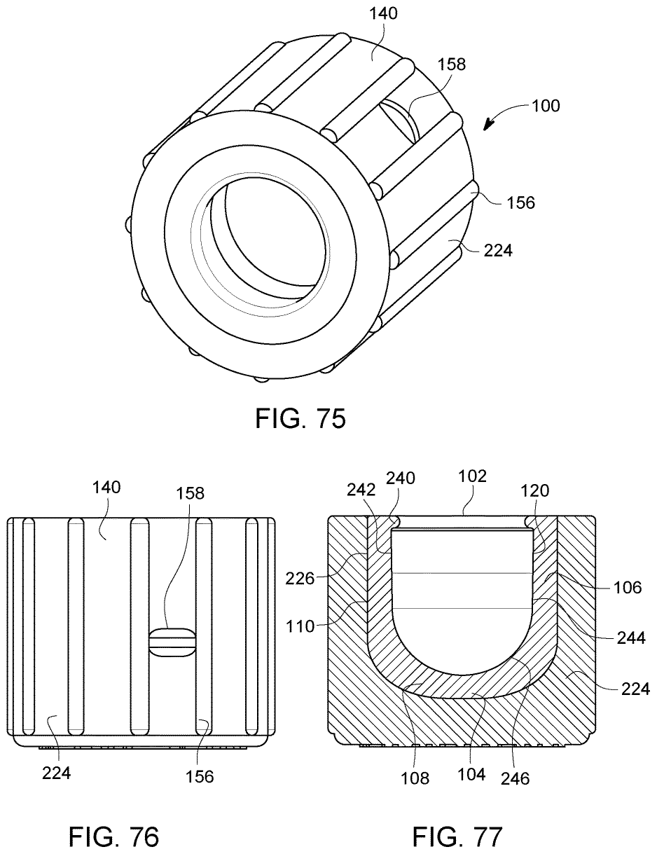

FIGS. 75-77 show a further illustrative embodiment of a disinfecting cap. The cap 100 may comprise an open end 102 and a closed end 104. A sidewall 106 may extend between the open end and closed end. An end wall 108 may be positioned at the closed end 104 of the cap. An outside surface 110 of the sidewall 106 may be generally cylindrical. The disinfection cap 100 may comprise a shell 224 that surrounds a least a portion of the sidewall 106 and the end wall 108. An inside surface 226 of the shell 224 may be positioned adjacent to the outside surface 110 of the sidewall 106. The inside surface of the shell may be adhered to the outside surface of the sidewall. An outer surface 140 of the shell 224 may comprise protuberances that extend from the outer surface such as columns 156 or tabs 158. The columns 156 and tabs 158 may be of various lengths, heights, and thicknesses and may vary in number and positioning as described elsewhere in this application. A lip 240 may extend inwardly from the cap interior surface 120 at or adjacent to the open end 102. The interior surface may have multiple sections, including a first section 242 adjacent the opening 102, a second, generally cylindrical section 244 adjacent the first section, and a third section 246 that transition from the sidewall 106 to the end wall 108. The third section may have a radius of curvature that may be between zero, such that the third section meets the end wall at a right angle, and a radius equal to the radius of the second section 244 such that the third section forms a hemisphere that is tangential with the second section. The shell 224 inside surface 226 may comprise similar sections that match, mirror, fit with, or otherwise reflect the cap sidewall sections.

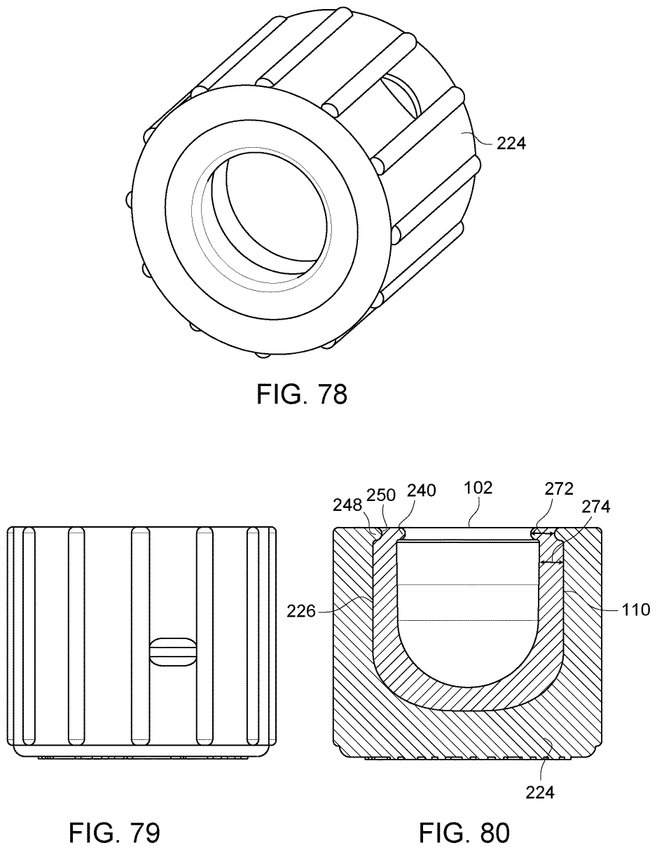

FIGS. 78-80 show a further illustrative embodiment of a disinfecting cap. The cap may comprise features similar to those described with respect to FIGS. 75-77 or elsewhere in this application. The shell 224 may comprise a material having a first durometer. The cap 100 may comprise a material having a second durometer that is softer than the first durometer. In addition or alternatively, the shell 224 may comprise a lip 248 that extends inwardly from the shell interior surface 226 at or adjacent to the open end 102. The lip 248 may engage a groove 250 formed in the outside surface 110 of the cap 100. The cap may comprise a corresponding lip 240 or may not. The cap may have a first thickness 272 at the lip 240 and a second thickness 274 below the lip 240. The first 272 and second 274 thicknesses may be the same such that the cap has a consistent thickness across the lip 240 and sidewall 226. In this manner, the geometry of the inside profile of the cap may correspond to the geometry of the inside profile of the shell. The shell 224 may be sized such that the opening created by the shell lip 248 has a diameter that is greater than a diameter of the port, and the cap 200 may be sized such that the opening created by the cap lip 240 is smaller than a diameter of the port such that the cap lip engages an outside surface of the port. The size and shape of the shell lip 248 may correspond in size or shape with the cap lip 240 or may comprise features of different size, scale, shape, or configuration.

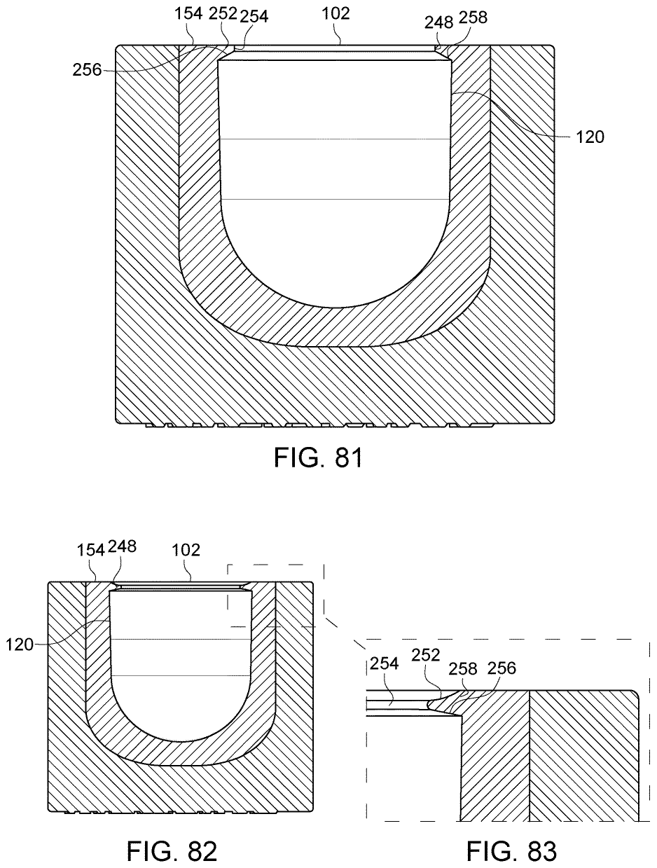

As illustrated in FIG. 81, a cap lip 248 may extend inwardly from the cap interior surface 120 at or adjacent to the open end 102. The lip may comprise a top section 252 that may be parallel to and/or continuous with an end surface 154 of the cap. An end section 254 of the lip may be curved or rectangular. A return section 256 of the lip may be angled relative to the top section 252 such that the lip is thicker at a base 258 than at the end section 254.

As illustrated in FIGS. 82-83, a cap lip 248 may extend inwardly from the cap interior surface 120 at or adjacent to the open end 102. The lip may comprise a top section 252 that is discontinuous with an end surface 154 of the cap. The top section may be generally straight but angled with respect to the cap end surface, and/or the top section may have curved surface. The curved surface may be concave or convex. An end section 254 of the lip may be curved or rectangular. A return section 256 of the lip may be angled relative to the top section 252 such that the lip is thicker at a base 258 than at the end section 254. The return section may be generally straight but angled with respect to the top section, and/or the return section may have curved surface. The curved surface may be concave or convex.

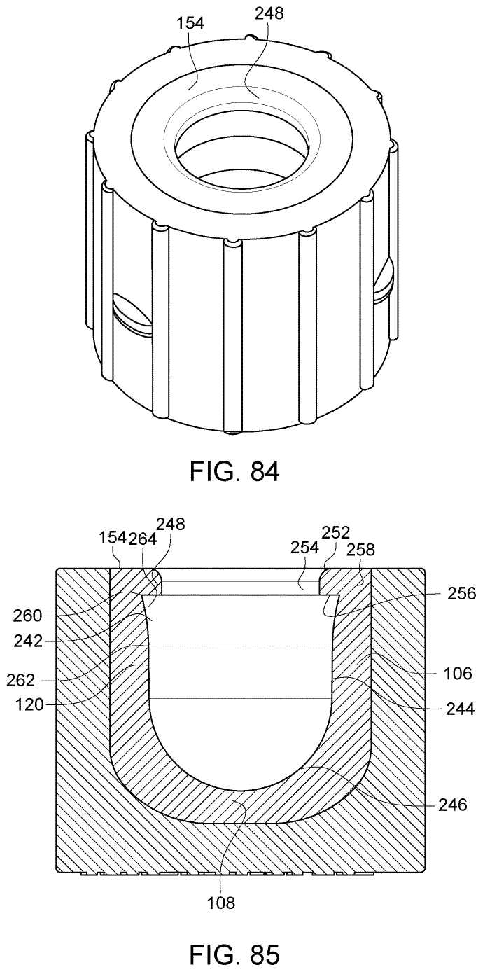

As illustrated in FIGS. 84-85, a cap lip 248 may extend inwardly from the cap interior surface 120 at or adjacent to the open end 102. The lip may comprise a top section 252 that is discontinuous with an end surface 154 of the cap. The top section may have curved surface that may create a continuous radius between the cap end surface 154 and the lip end section 254. A return section 256 of the lip may be parallel to the cap end surface 154. The cap interior surface 120 may have multiple sections, including a first section 242 adjacent the opening 102, a second, generally cylindrical section 244 adjacent the first section, and a third section 246 that transition from the sidewall 106 to the end wall 108. The first section 242 may be curved or angled outwardly with respect to the second section 244 such that the cap sidewall 106 is narrower at an upper end 260 than at a lower end 262. Accordingly, the lip return section 256 may extend outwardly beyond the lip top section 252 thereby creating an undercut 264 adjacent the lip 248.

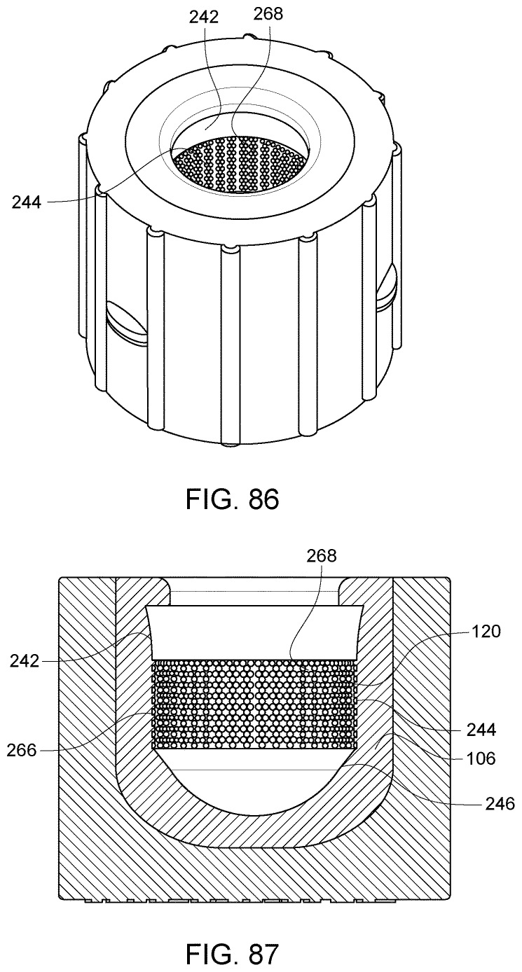

FIGS. 86-87 show a further illustrative embodiment of a disinfecting cap. The cap may comprise features similar to those described with respect to FIGS. 84-85 or elsewhere in this application. In addition or alternatively, the second section 244 of the cap sidewall 106 may comprise texturing 268 such as knurling, dimpling, or the like while the first section 242 and/or third section 246 may not include texturing 268. For example, a series of dimples 266 or holes may be formed in the inside surface 120 extending into the sidewall 106. The dimples 266 may be formed in a repeating pattern covering part or all of the second section 244. Alternatively, the texturing 268 may comprise protrusions that extend from a recessed portion of the second sidewall section 244. The texturing 268 may affect a press or friction fit between the cap interior surface 120 and a female luer 6802 of a fitting such as Y-site 6800. The size shape and configuration of the texturing 268 or dimples 266 may be adjusted to increase or decrease deformation, insertion force, retention force, or other parameters. The texturing 268 or dimples 266 may also form one or more channels that allow fluid movement between a position in front of and a position behind the luer threads 6804 as the threads are inserted into the cap.

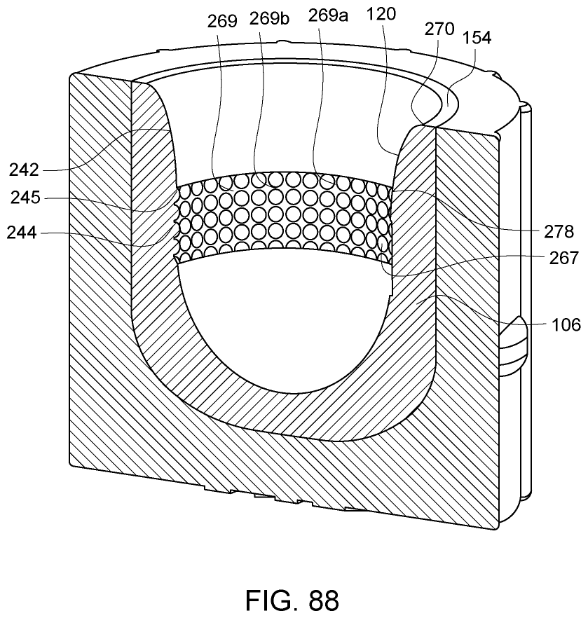

FIG. 88 shows a further illustrative embodiment of a disinfecting cap. The cap may comprise features similar to those described with respect to FIGS. 84-87 or elsewhere in this application. In addition or alternatively, the first section 242 of the cap sidewall 106 may extend from the second section 244 to the cap end surface 154 in an angled or curved manner such that a thickness of the sidewall 106 as greater at a lower end 278 of the first section than at the intersection 270 between the sidewall inside surface 120 and the cap end surface 154. The first section may be generally straight but angled with respect to the second section, and/or the first section may have curved surface. The curved surface may be concave or convex.

As illustrated in FIG. 88, the second section 244 of the cap sidewall 106 may include texturing that comprises a plurality of protrusions 267 that extend from the second sidewall section 244. The protrusions may be arranged in a repeated pattern. The second section 244 may comprise a recessed portion 245. The protrusions 267 may be circular and may be domed or hemispherical. The pattern of protrusions 267 may create channels 269 that extend between the protrusions. The channels 269 may extend circumferentially or longitudinally along the sidewall second portion 244. The channels 269 may allow longitudinal fluid movement between a position in front of and a position behind the luer threads 6804 as the threads are inserted into the cap. In particular, longitudinally extending channels 269a may promote such longitudinal movement of fluid. Circumferential channels 269b may allow for or promote circumferential movement of fluid such that the fluid moves around the entire circumference of the lure threads 6804 or other port inserted into the cap.

Although a few embodiments have been described in detail above, other modifications are possible. For instance, any of the embodiments described above may be sized and scaled for a particular medical implement, such as a stethoscope or otoscope. Other embodiments may be within the scope of the following claims.

This application provides a description of various implementations and embodiments of a device for cleaning medical implements. The various embodiments have been described as having a variety of features. It will be understood by one of ordinary skill in the art that features of the various embodiments are intended to be interchangeable, and features described in the context of one embodiment may be implemented in conjunction with a device having the features and structure of another embodiment.

What is claimed is:

1. A disinfecting cap for attachment to a port of a medical implement, the cap comprising:
a cap comprising:
an open end,
a closed end,
a sidewall extending between the open end and the closed end, the sidewall comprising a sidewall outside surface and a sidewall inside surface, and
an end wall positioned at the closed end;
a shell that surrounds at least a portion of the sidewall, the shell comprising a shell inside surface positioned adjacent to the sidewall outside surface,
wherein the sidewall inside surface comprises:
a first section adjacent the cap open end,
a second section adjacent the first section and spaced apart from the cap open end, and
a third section that transitions from the sidewall to the end wall;
wherein the second section comprises a plurality of protrusions arranged in a pattern defining a plurality of channels extending between one or more of the plurality of protrusions;
wherein one or more of the plurality of protrusions contacts a surface of the port; and
wherein the plurality of channels is configured such that a fluid disposed in the cap flows along one or more of the plurality of channels when the port is inserted into the cap.

2. The disinfecting cap of claim 1 wherein the shell inside surface is adhered to the sidewall outside surface.

3. The disinfecting cap of claim 1 wherein the second section is cylindrical.

4. The disinfecting cap of claim 3 wherein the third section transitions between the sidewall and the end wall.

5. The disinfecting cap of claim 4 wherein the third section comprises a radius of curvature.

6. The disinfecting cap of claim 5 wherein the radius of curvature is zero, such that the third section meets the end wall at a right angle.

7. The disinfecting cap of claim 5 wherein the radius of curvature is equal to a radius of the cylindrical second section such that the third section forms a hemisphere that is tangential with the cylindrical second section.

8. The disinfecting cap of claim 5 wherein the radius of curvature is between zero and a radius that is equal to a radius of the cylindrical second section.

9. The disinfecting cap of claim 1 wherein one or more of the plurality of protrusions contacts a thread of the port.

10. The disinfecting cap of claim 9 wherein one or more of the plurality of protrusions engages the port thread with a friction fit.

11. The disinfecting cap of claim 10 wherein one or more of the plurality of channels extends longitudinally along the sidewall second section.

12. The disinfecting cap of claim 11 wherein one or more of the plurality of channels is configured such that the fluid flows longitudinally between a position on a first side of the port thread and a position on a second side of the port thread.

13. The disinfecting cap of claim 10 wherein one or more of the plurality of channels extends circumferentially around the sidewall second section.

14. The disinfecting cap of claim 13 wherein one or more of the plurality of channels is configured such that the fluid flows circumferentially around an entire circumference of the port.

15. The disinfecting cap of claim 14 wherein the circumferential fluid flow wets a thread on a first side of the port and a thread on a second side of the port.

16. The disinfecting cap of claim 1 wherein one or more protrusions of the plurality of protrusions are circular.

17. The disinfecting cap of claim 16 wherein one or more protrusions of the plurality of protrusions are domed.

18. The disinfecting cap of claim 16 wherein one or more protrusions of the plurality of protrusions are hemispherical.

19. A disinfecting cap for attachment to a port of a medical implement, the cap comprising:
a cap comprising:
an open end,
a closed end,
a sidewall extending between the open end and the closed end, the sidewall comprising a sidewall outside surface and a sidewall inside surface, and
an end wall positioned at the closed end;
a shell that surrounds at least a portion of the sidewall, the shell comprising a shell inside surface positioned adjacent to the sidewall outside surface,
wherein the sidewall inside surface comprises:
a first section adjacent the cap open end,
a second section adjacent the first section and spaced apart from the cap open end, and
a third section that transitions from the sidewall to the end wall; and
wherein the second section comprises a plurality of protrusions arranged in a pattern defining a plurality of longitudinal channels extending between one or more of the plurality of protrusions and a plurality of circumferential channels extending between one or more of the plurality of protrusions;
wherein one or more of the plurality of protrusions contacts a surface of the port; and
wherein the plurality of channels is configured such that a fluid disposed in the cap flows along the plurality of channels when the port is inserted into the cap.

20. The disinfecting cap of claim 11 wherein:
one or more of the plurality of protrusions contacts a thread of the port;
one or more of the plurality of longitudinal channels is configured such that the fluid flows longitudinally between a position on a first side of the port thread and a position on a second side of the port thread; and
one or more of the plurality of circumferential channels is configured such that the fluid flows circumferentially around an entire circumference of the port.
